(19) United States
(12) Patent Application Publication
(10) Pub. No.: US 2025/0391572 A1
(43) Pub. Date: Dec. 25, 2025

(54) Multi-Modal Health Scoring and Recommendation Generation System and Method Thereof

(71) Applicant: CARDIAI TECHNILOGIES, Calgary (CA)

(72) Inventor: Anmol Singh Kapoor, Calgary (CA)

(21) Appl. No.: 19/224,842

(22) Filed: Jun. 1, 2025

Related U.S. Application Data
(60) Provisional application No. 63/661,640, filed on Jun. 19, 2024.

(51) Int. Cl. G16H 50/30; A61B 5/00; G16B 30/00

(52) U.S. Cl. CPC G16H 50/30; A61B 5/486; A61B 5/7246; A61B 5/7264; A61B 5/7275; A61B 5/7435; A61B 5/7465; A61B 5/7475; G16B 30/00; A61B 2503/10

(57) ABSTRACT

The present disclosure relates to a multi-modal health scoring and recommendation generation system and method thereof (100) comprising a data acquisition unit (102) for acquiring a plurality of health and performance data from internal or external sources, a communication network (104) operatively connected to the data acquisition unit (102), a processing unit (106) operatively connected to the data acquisition unit (102) through the communication network (104), the processing unit (106) comprises; a health profiling module (108), a scoring module (110), an artificial intelligence recommendation module (112), a feedback integration module (114), a telemedicine integration module (116), a dashboard module (118), a database unit (120) operatively connected to the processing unit (106), a user device (122) operatively connected to the processing unit (106) through the communication network (104), a user interface (124) disposed within the user device (122) and configured for enabling access to visualizations, recommendations, and profile information.

### FIELD OF THE INVENTION

Embodiments of the present invention relate to the field of digital health analytics and specifically relates to a multi-modal health scoring and recommendation generation system and method thereof.

### DESCRIPTION OF THE RELATED ART

The present invention is offering each individual a deeply customized experience tailored to their unique health conditions, habits, and goals. This personalized approach is helping users feel more understood and motivated in their wellness journey. It is not offering generic advice but is focusing on what truly matters to the individual. The system is continuously adapting to changing user needs, helping them make better lifestyle choices. This ongoing personalization is improving user engagement and satisfaction.

Unlike services that focus on a single health aspect, this invention is addressing the complete picture of a person's wellness. It is taking into account multiple aspects of lifestyle, diet, and activity to provide comprehensive support. This holistic nature is enabling users to balance physical activity, mental well-being, and nutrition. Users are being empowered with meaningful recommendations to live healthier and more balanced lives. The invention is encouraging long-term lifestyle improvements instead of temporary fixes.

The invention is helping users better understand their own health by offering meaningful insights and scores. This awareness is empowering individuals to take more responsibility for their wellness and make informed decisions. It is providing easy-to-understand feedback that promotes healthy changes in a user-friendly way. Users are gaining clarity on how their daily habits are affecting their well-being. This sense of control is making users feel more confident and proactive in managing their health.

The invention is helping users stay motivated by showing progress in their health journey over time. It is encouraging consistent improvement by reflecting positive lifestyle choices in the form of updated scores or recommendations. This real-time feedback is creating a sense of achievement, helping users stay committed to their goals. People are more likely to stick to routines when they see their efforts paying off. It is creating a cycle of motivation that is sustaining healthy behavior.

The invention is fitting easily into people's daily routines, making healthy living feel natural rather than forced. Its design is focusing on ease of use, minimizing the need for users to change their habits drastically. The suggestions and updates are being delivered in a way that aligns with how people live today. Users are not overwhelmed with information but are receiving helpful and timely guidance. This seamless integration is helping people maintain healthy habits more sustainably.

Many existing wellness platforms are providing broad and one-size-fits-all recommendations that lack relevance to the individual. This generic approach is often failing to inspire user trust or action. People are not feeling personally understood, which is reducing motivation. Users often ignore suggestions because they do not align with their specific needs. The lack of customization is making the experience less engaging and less helpful in the long term.

Most current wellness tools are focusing on a single health factor without considering the larger picture. This fragmented view is forcing users to juggle multiple apps or services for different health areas. It is difficult for users to make meaningful changes when their information is scattered across platforms. The lack of a unified approach is leading to confusion and inefficiency. As a result, users are feeling unsupported and overwhelmed by mixed signals.

Many existing systems are only offering static recommendations without adapting to ongoing changes in users' lifestyles. Without regular updates or feedback, users are losing interest quickly. The lack of ongoing interaction is leading to poor engagement and low retention. People are dropping off because they feel the platform is no longer relevant to their evolving needs. This disconnect is preventing users from reaching their long-term health goals.

Several health platforms are overwhelming users with too many details or confusing interfaces. This complexity is discouraging users from continuing with the system. When people find it difficult to understand or follow, they simply give up. The learning curve is making it hard for users to benefit fully from the platform. Simplicity and clarity are often missing, leading to frustration. Users are seeking guidance, not stress, and overly complex systems are failing in this regard.

Several health platforms are overwhelming users with too many details or confusing interfaces. This complexity is discouraging users from continuing with the system. When people find it difficult to understand or follow, they simply give up. The learning curve is making it hard for users to benefit fully from the platform. Simplicity and clarity are often missing, leading to frustration. Users are seeking guidance, not stress, and overly complex systems are failing in this regard.

In conclusion, the present invention is standing out as a powerful and user-focused innovation that is transforming the wellness experience into something personal, engaging, and sustainable. By overcoming the limitations of existing solutions, it is empowering individuals to take ownership of their health with clarity and confidence. The system is helping users make meaningful lifestyle changes that last. This invention is reshaping the future of personal health by promoting balance, motivation, and continuous improvement in daily living.

Thus, there is a need of a multi-modal health scoring and recommendation generation system and method thereof.

Therefore, the present invention provides a multi-modal health scoring and recommendation generation system and method thereof.

### SUMMARY OF THE INVENTION

Embodiments of the present invention relate to a multi-modal health scoring and recommendation generation system, the system. The system comprising a data acquisition unit for acquiring a plurality of health and performance data from internal or external sources. The system also comprises a communication network operatively connected to the data acquisition unit and configured for enabling data exchange between the data acquisition unit and other components of the system. The system also comprises a processing unit operatively connected to the data acquisition unit through the communication network, the processing unit comprises; a health profiling module configured for generating a user profile based on correlation of the acquired data, a scoring module configured for generating one or more health-related scores based on the user profile, an artificial intelligence recommendation module configured for producing personalized recommendation outputs, a feedback integration module configured for updating the one or more health-related scores and the personalized recommendation outputs based on real-time data streams, a telemedicine integration module configured for facilitating remote clinical interaction and risk assessment, a dashboard module configured for generating interactive visualizations of the user profile, the one or more health-related scores, and the personalized recommendation outputs. The system also comprises a database unit operatively connected to the processing unit and configured for securely storing the acquired data, the user profile, the one or more health-related scores, the personalized recommendation outputs, and related usage history. The system also comprises a user device operatively connected to the processing unit through the communication network, the user device being configured for receiving user input and delivering output to the user. The system also comprises a user interface disposed within the user device and configured for enabling access to visualizations, recommendations, and profile information.

In accordance with an embodiment of the present invention, the data acquisition unit comprises a genomic data interface configured for receiving genomic data including whole genome sequencing or exome sequencing files from external laboratory systems.

In accordance with an embodiment of the present invention, the data acquisition unit comprises a wearable device interface configured for receiving real-time activity and physiological data from one or more external wearable tracking devices.

In accordance with an embodiment of the present invention, the processing unit further comprises a polygenic risk analysis module configured for generating a risk score for cardiac disease based on a plurality of genetic markers.

In accordance with an embodiment of the present invention, the processing unit further comprises a benchmarking module configured for comparing the user profile with stored profiles of elite athletes.

In accordance with an embodiment of the present invention, the health profiling module comprises a behavioural history analysis module configured for incorporating longitudinal lifestyle data into the user profile based on historical behaviour patterns.

In accordance with an embodiment of the present invention, the scoring module further comprises a trait-mapping engine for associating user data with performance, recovery, nutrition, and injury-resilience traits.

In accordance with an embodiment of the present invention, the artificial intelligence recommendation module comprises a clustering logic engine for identifying user cohorts based on genomic and lifestyle similarity.

In accordance with an embodiment of the present invention, the artificial intelligence recommendation module further comprises a rules-based inference engine trained on outcome data for generating risk mitigation suggestions.

In accordance with an embodiment of the present invention, feedback integration module further comprises a continuous data listener configured for detecting behavioural deviations from predicted activity patterns.

In accordance with an embodiment of the present invention, telemedicine integration module further comprises a triage prioritization engine for automatically classifying users based on risk levels for clinical review.

In accordance with an embodiment of the present invention, telemedicine integration module further comprises a secure interface for real-time voice, video, or text-based communication between the user and a remote healthcare provider.

In accordance with an embodiment of the present invention, dashboard module comprises a dynamic visualization builder configured for generating time-series trends for each score over a configurable time window.

In accordance with an embodiment of the present invention, the user interface comprises an interactive feedback panel allowing users to submit responses or preferences for improving recommendation quality.

In accordance with an embodiment of the present invention, user device comprises a biometric sensor unit configured for locally capturing one or more physiological parameters including heart rate, skin temperature, or oxygen saturation.

In accordance with an embodiment of the present invention, database unit comprises a data encryption layer for secure long-term storage of genomic and health scoring information.

In accordance with an embodiment of the present invention, processing unit further comprises a learning module configured for adapting scoring and recommendation behaviour based on aggregated anonymous user data.

In accordance with an embodiment of the present invention, processing unit further comprises a context recognition module configured for adjusting recommendations based on environmental or temporal variables detected from the user device.

Another embodiment of the present invention relates to a multi-modal health scoring and recommendation generation method. The method includes acquiring a plurality of health and performance data from internal or external sources using a data acquisition unit. The method also includes transmitting the acquired data through a communication network to a processing unit. The method also includes generating a user profile within a health profiling module by correlating the acquired data. The method also includes computing one or more health-related scores within a scoring module based on the user profile. The method also includes producing personalized recommendation outputs within an artificial intelligence recommendation module based on the one or more health-related scores. The method also includes receiving real-time data streams through the data acquisition unit and transmitting the real-time data to the processing unit through the communication network. The method also includes updating the one or more health-related scores and the personalized recommendation outputs within a feedback integration module using the real-time data streams. The method also includes facilitating remote clinical interaction and risk assessment through a telemedicine integration module connected to the artificial intelligence recommendation module and the scoring module. The method also includes presenting visualizations of the user profile, the one or more health-related scores, and the personalized recommendation outputs on a dashboard module through a user interface disposed within a user device. The method also includes storing the acquired data, the user profile, the one or more health-related scores, and the personalized recommendation outputs in a database unit.

In accordance with an embodiment of the present invention, the method further comprises the step of performing a benchmarking operation within the processing unit, wherein the benchmarking operation is comparing the user profile and the one or more health-related scores against stored profiles of elite performers to adjust the personalized recommendation outputs.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 illustrates a block diagram of the multi-modal health scoring and recommendation generation system and method thereof, in accordance with an embodiment of the present invention;

FIG. 2 illustrates a flowchart of a multi-modal health scoring and recommendation generation system, in accordance with an embodiment of the present invention;

FIG. 3 illustrates a flowchart of a multi-modal health scoring and recommendation generation method, in accordance with an embodiment of the present invention;

FIG. 4 illustrates a recommendation workflow of the health scoring and recommendation generation system, in accordance with an embodiment of the present invention;

FIG. 5 illustrates a screenshot of the lifestyle score algorithm of a health scoring and recommendation generation system, in accordance with an embodiment of the present invention;

FIG. 6 illustrates a screenshot of the activity updating of a health scoring and recommendation generation system, in accordance with an embodiment of the present invention.

It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The following brief definition of terms shall apply throughout the present invention

The terms “determining”, “measuring”, “evaluating”, “assessing,” “assaying,” and “analyzing” can be used interchangeably herein to refer to any form of measurement, and include determining if an element is present or not. (e.g., detection). These terms can include both quantitative and/or qualitative determinations. Assessing may be relative or absolute.

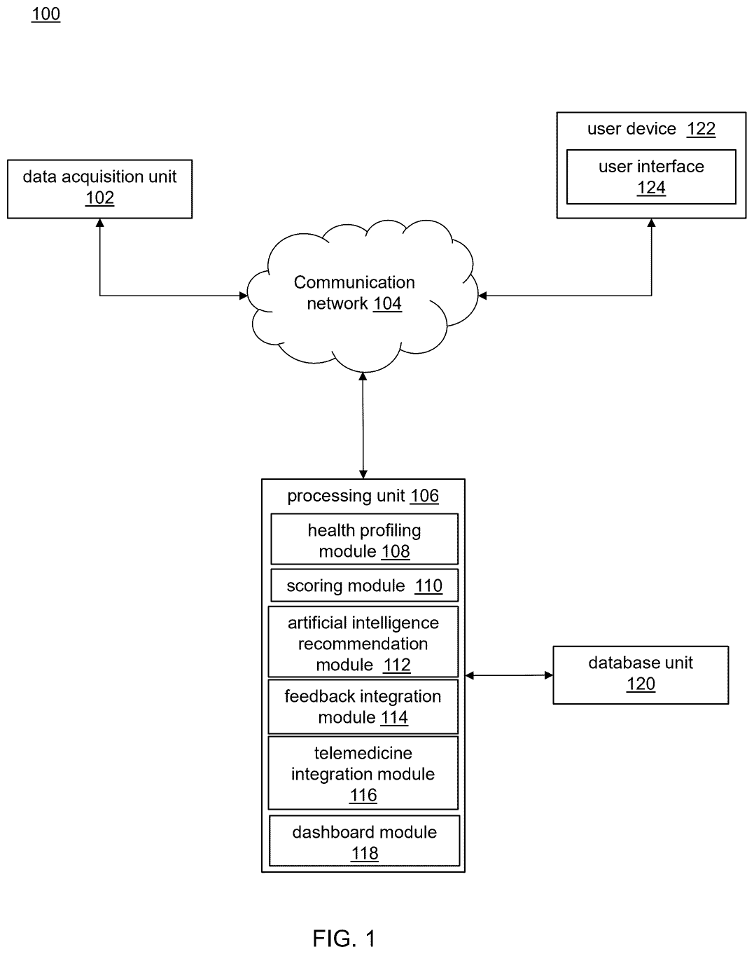

FIG. 1 illustrates a block diagram of the multi-modal health scoring and recommendation generation system and method thereof 100, in accordance with an embodiment of the present invention.

The system 100 may comprise a data acquisition unit 102 for acquiring a plurality of health and performance data from internal or external sources. The system 100 may include a communication network 104 operatively connected to the data acquisition unit 102 and configured for enabling data exchange between the data acquisition unit 102 and other components of the system. The system 100 may include a processing unit 106 operatively connected to the data acquisition unit 102 through the communication network 104, the processing unit 106 comprises a health profiling module 108 configured for generating a user profile based on correlation of the acquired data, a scoring module 110 configured for generating one or more health-related scores based on the user profile, an artificial intelligence recommendation module 112 configured for producing personalized recommendation outputs, a feedback integration module 114 configured for updating the one or more health-related scores and the personalized recommendation outputs based on real-time data streams, a telemedicine integration module 116 configured for facilitating remote clinical interaction and risk assessment, a dashboard module 118 configured for generating interactive visualizations of the user profile, the one or more health-related scores, and the personalized recommendation outputs. The system 100 may include a database unit 120 operatively connected to the processing unit 106 and configured for securely storing the acquired data, the user profile, the one or more health-related scores, the personalized recommendation outputs, and related usage history. The system 100 may include a user device 122 operatively connected to the processing unit 106 through the communication network 104, the user device 122 being configured for receiving user input and delivering output to the user. The system 100 may include a user interface 124 disposed within the user device 122 and configured for enabling access to visualizations, recommendations, and profile information.

The data acquisition unit 102 comprises a genomic data interface configured for receiving genomic data including whole genome sequencing or exome sequencing files from external laboratory systems.

The data acquisition unit 102 comprises a wearable device interface configured for receiving real-time activity and physiological data from one or more external wearable tracking devices.

The processing unit 106 further comprises a polygenic risk analysis module configured for generating a risk score for cardiac disease based on a plurality of genetic markers.

The processing unit 106 further comprises a benchmarking module configured for comparing the user profile with stored profiles of elite athletes.

The health profiling module 108 comprises a behavioral history analysis module configured for incorporating longitudinal lifestyle data into the user profile based on historical behavior patterns.

The scoring module 110 further comprises a trait-mapping engine for associating user data with performance, recovery, nutrition, and injury-resilience traits.

The artificial intelligence recommendation module 112 comprises a clustering logic engine for identifying user cohorts based on genomic and lifestyle similarity.

The artificial intelligence recommendation module 112 further comprises a rules-based inference engine trained on outcome data for generating risk mitigation suggestions.

The feedback integration module 114 further comprises a continuous data listener configured for detecting behavioral deviations from predicted activity patterns.

The telemedicine integration module 116 further comprises a triage prioritization engine for automatically classifying users based on risk levels for clinical review.

The telemedicine integration module 116 further comprises a secure interface for real-time voice, video, or text-based communication between the user and a remote healthcare provider.

The dashboard module 118 comprises a dynamic visualization builder configured for generating time-series trends for each score over a configurable time window.

The user interface 124 comprises an interactive feedback panel allowing users to submit responses or preferences for improving recommendation quality.

The user device 122 comprises a biometric sensor unit configured for locally capturing one or more physiological parameters including heart rate, skin temperature, or oxygen saturation.

The database unit 120 comprises a data encryption layer for secure long-term storage of genomic and health scoring information.

The processing unit 106 further comprises a learning module configured for adapting scoring and recommendation behaviour based on aggregated anonymous user data.

The processing unit 106 further comprises a context recognition module configured for adjusting recommendations based on environmental or temporal variables detected from the user device 122.

The method 100 may comprise acquiring a plurality of health and performance data from internal or external sources using a data acquisition unit 102. The method 100 may include transmitting the acquired data through a communication network 104 to a processing unit 106. The method 100 may also include generating a user profile within a health profiling module 108 by correlating the acquired data. The method 100 may also include computing one or more health-related scores within a scoring module 110 based on the user profile. The method 100 may also include producing personalized recommendation outputs within an artificial intelligence recommendation module 112 based on the one or more health-related scores. The method 100 may also include receiving real-time data streams through the data acquisition unit 102 and transmitting the real-time data to the processing unit 106 through the communication network 104. The method 100 may also include updating the one or more health-related scores and the personalized recommendation outputs within a feedback integration module 114 using the real-time data streams. The method 100 may also include facilitating remote clinical interaction and risk assessment through a telemedicine integration module 116 connected to the artificial intelligence recommendation module 112 and the scoring module 110. The method 100 may also include presenting visualizations of the user profile, the one or more health-related scores, and the personalized recommendation outputs on a dashboard module 118 through a user interface 124 disposed within a user device 122. The method 100 may also include storing the acquired data, the user profile, the one or more health-related scores, and the personalized recommendation outputs in a database unit 120.

The method further comprises the step of performing a benchmarking operation within the processing unit 106, wherein the benchmarking operation is comparing the user profile and the one or more health-related scores against stored profiles of elite performers to adjust the personalized recommendation outputs.

The data acquisition unit 102 acquires a plurality of health and performance data from internal and external sources including but not limited to genomic data, cardiac gene panel data, microbiome data, lifestyle and health questionnaire responses, demographic and clinical history information, and real-time physiological signals from wearable tracking devices. The data acquisition unit 102 comprises a genomic data interface that receives whole genome sequencing files and exome sequencing files generated from biological samples including saliva and blood, as processed by external laboratory systems. The data acquisition unit 102 further includes a wearable device interface for real-time ingestion of parameters such as step count, heart rate, sleep duration, oxygen saturation, skin temperature, and movement patterns, enabling continuous behavioral and physiological monitoring. The data acquisition unit 102 also receives structured questionnaire responses capturing user-reported lifestyle elements including physical activity level, dietary practices, body composition, medication intake, family history, and risk exposure. The data acquisition unit 102 supports secure integration with healthcare systems to acquire electronic medical records or clinical history relevant to the user. The data acquisition unit 102 is responsible for normalizing and transmitting the raw and structured inputs through the communication network 104 to the processing unit 106 for downstream operations including profiling, scoring, and personalized recommendation generation.

The communication network 104 enables seamless and secure transmission of all acquired health and performance data from the data acquisition unit 102 to the processing unit 106 and coordinates bi-directional data flow between all other components of the multi-modal health scoring and recommendation generation system. The communication network 104 supports distributed data exchange using secure internet protocols and connects the user device 122, the processing unit 106, and the database unit 120 in a synchronized loop. The communication network 104 enables continuous streaming of real-time physiological metrics collected by wearable tracking devices to the feedback integration module 114 and facilitates the transmission of questionnaire data and laboratory-derived genomic sequences to the health profiling module 108. The communication network 104 supports cloud-based communication architecture enabling modular execution of the scoring module 110 and artificial intelligence recommendation module 112. The communication network 104 facilitates real-time interactions between the user interface 124 and the dashboard module 118, allowing updated visualizations and recommendations to be presented to the user device 122 without delay. The communication network 104 also allows the telemedicine integration module 116 to transmit and receive remote clinical consultation data including voice, video, or text communication sessions. The communication network 104 ensures encryption, latency minimization, and uninterrupted connectivity across all data exchange points.

The processing unit 106 serves as the computational core of the multi-modal health scoring and recommendation generation system and executes all software modules necessary for data transformation, scoring, recommendation generation, and clinical interaction. The processing unit 106 receives normalized health and performance data from the data acquisition unit 102 through the communication network 104 and coordinates the functioning of the health profiling module 108, scoring module 110, artificial intelligence recommendation module 112, feedback integration module 114, telemedicine integration module 116, and dashboard module 118. The processing unit 106 processes whole genome sequencing data, polygenic cardiac risk markers, demographic variables, activity data, and lifestyle attributes in real time. The processing unit 106 applies adaptive learning logic to adjust its scoring and recommendation pathways using pattern recognition, trait-mapping, benchmarking comparisons with elite athlete profiles, and population-derived polygenic models. The processing unit 106 also supports modular expansion, allowing additional algorithms or modules such as a learning module or context recognition module to be executed concurrently. The processing unit 106 maintains secure access to the database unit 120 for retrieval and storage of user profiles, historical data, score progression, and recommendation history. The processing unit 106 ensures continuous operation of the entire system infrastructure.

The health profiling module 108 generates a multi-dimensional user profile by correlating a plurality of acquired health and performance data received from the data acquisition unit 102. The health profiling module 108 processes genetic information from whole genome sequencing and exome sequencing files, polygenic cardiac panels, demographic variables including age, gender, ethnicity, and lifestyle data collected through health and activity questionnaires. The health profiling module 108 integrates behavioral and physiological indicators such as sleep quality, activity patterns, nutritional intake, and self-reported health conditions. The health profiling module 108 includes a behavioral history analysis module that compiles and updates longitudinal data patterns to reflect user lifestyle trends over time. The health profiling module 108 aligns collected inputs with defined trait categories including endurance, metabolic efficiency, stress resilience, injury susceptibility, and recovery rate. The health profiling module 108 prepares a structured and annotated user profile to be interpreted by the scoring module 110 for subsequent scoring. The health profiling module 108 also prepares data for downstream comparison with elite performer datasets and for phenotype-genotype clustering tasks in the artificial intelligence recommendation module 112. The health profiling module 108 enables high-resolution personalization across all dimensions of user health and performance characteristics.

The scoring module 110 generates one or more health-related scores using the structured user profile prepared by the health profiling module 108. The scoring module 110 applies algorithmic weighting models, polygenic scoring methods, and time-based decay functions to produce a lifestyle score, a bio fit score, and a polygenic cardiac risk score. The scoring module 110 includes a trait-mapping engine that associates user data with distinct categories including endurance capacity, metabolic function, cardiovascular resilience, musculoskeletal integrity, recovery ability, and psychological readiness. The scoring module 110 references internal databases of elite athlete genomic and performance benchmarks to compute comparative scores. The scoring module 110 integrates environmental and social context elements into the scoring outputs and assigns variable weights based on user category, goal preference, and risk factor intensity. The scoring module 110 continuously updates scores in response to real-time data from the feedback integration module 114 and integrates corrections based on deviations between expected and observed user outcomes. The scoring module 110 enables multi-layered, phenotype-aware, and context-specific evaluations of user health across physical, nutritional, mental, and cardiac domains. The scoring module 110 provides foundational metrics that guide the artificial intelligence recommendation module 112.

The artificial intelligence recommendation module 112 generates personalized recommendation outputs based on the scores received from the scoring module 110. The artificial intelligence recommendation module 112 applies dynamic learning algorithms, clustering techniques, and rules-based inference to create individualized nutrition plans, exercise protocols, mental wellness interventions, and cardiac clearance decisions. The artificial intelligence recommendation module 112 processes multi-modal inputs and historical outcomes to segment users into genomic and lifestyle similarity groups using a clustering logic engine. The artificial intelligence recommendation module 112 also integrates a rules-based inference engine that applies learned outcome patterns to adjust recommendations for nutrition, recovery, training intensity, and stress modulation. The artificial intelligence recommendation module 112 evaluates the comparative genotype-phenotype mappings between the user profile and elite athlete reference data to calibrate training regimens and risk thresholds. The artificial intelligence recommendation module 112 dynamically adapts its output in real time using updated information from the feedback integration module 114. The artificial intelligence recommendation module 112 generates outputs that are streamed to the dashboard module 118 for visualization and to the telemedicine integration module 116 for remote consultation delivery. The artificial intelligence recommendation module 112 ensures contextual personalization and evidence-driven recommendation flow.

The feedback integration module 114 continuously updates the one or more health-related scores and the personalized recommendation outputs based on incoming real-time data streams acquired by the data acquisition unit 102 and transmitted through the communication network 104. The feedback integration module 114 processes physiological and behavioral data including heart rate, sleep duration, step count, movement variability, skin temperature, and oxygen saturation received from wearable tracking devices interfaced with the wearable device interface of the data acquisition unit 102. The feedback integration module 114 includes a continuous data listener that detects deviations from expected user behavior patterns and applies correction inputs to adjust ongoing scoring processes within the scoring module 110 and recommendation logic within the artificial intelligence recommendation module 112. The feedback integration module 114 ensures that all changes in user lifestyle, physiological condition, and compliance levels are reflected promptly in the scoring output and the subsequent recommendation stream. The feedback integration module 114 supports bi-directional data flows that reinforce adaptive personalization and prevent outdated or static recommendation patterns. The feedback integration module 114 interacts directly with the processing unit 106 to enable continuous learning by sharing feedback records with the learning module, when present. The feedback integration module 114 maintains real-time performance optimization for the overall system.

The telemedicine integration module 116 facilitates remote clinical interaction and personalized risk assessment by connecting users and healthcare professionals through the communication network 104. The telemedicine integration module 116 accesses the user profile, one or more health-related scores, and personalized recommendation outputs generated by the health profiling module 108, scoring module 110, and artificial intelligence recommendation module 112 respectively. The telemedicine integration module 116 includes a triage prioritization engine that classifies users based on risk level, urgency, and medical history to streamline virtual intervention workflows. The telemedicine integration module 116 supports secure voice, video, and messaging communication features, enabling real-time consultations between users and licensed clinical personnel through the user device 122. The telemedicine integration module 116 allows healthcare professionals to provide diagnostic opinions, deliver sport-specific medical guidance, and intervene on high-risk physiological alerts such as arrhythmia signals inferred from cardiac gene panel analysis. The telemedicine integration module 116 also provides tools for physicians to modify ongoing recommendation protocols generated by the artificial intelligence recommendation module 112. The telemedicine integration module 116 maintains a traceable log of every virtual interaction and shares relevant updates with the database unit 120 for secure archival and audit readiness.

The dashboard module 118 generates interactive visualizations of the user profile, the one or more health-related scores, and the personalized recommendation outputs for presentation through the user interface 124 on the user device 122. The dashboard module 118 integrates scoring outputs from the scoring module 110, recommendation pathways from the artificial intelligence recommendation module 112, and trend data from the feedback integration module 114. The dashboard module 118 includes a dynamic visualization builder that creates time-series representations of scores and behaviors across configurable time windows. The dashboard module 118 allows for goal tracking, trend recognition, and comparative progress analysis based on reference benchmarks. The dashboard module 118 provides customizable views for displaying categorized insights such as nutrition balance, exercise compliance, mental wellness indicators, recovery cycles, and cardiac risk variation. The dashboard module 118 receives updated inputs from the processing unit 106 in real time and refreshes visual elements on the user interface 124 without requiring user prompts. The dashboard module 118 enhances transparency, comprehension, and motivation by delivering graphical narratives that reflect the evolving state of user health. The dashboard module 118 acts as the primary output visualization layer that supports both user engagement and clinician review via the telemedicine integration module 116.

The database unit 120 securely stores all acquired data, the structured user profile, the one or more health-related scores, the personalized recommendation outputs, and any historical usage logs associated with user activity, compliance, and telemedicine interactions. The database unit 120 connects directly to the processing unit 106 via the communication network 104 and supports persistent storage architecture with encrypted access protocols. The database unit 120 archives raw genomic files including whole genome sequencing and cardiac gene panels, microbiome outputs, structured lifestyle questionnaire data, clinical history records, and processed feature sets used for profiling. The database unit 120 also stores polygenic cardiac risk scores, bio fit scores, comparative metrics against elite athlete profiles, and updates from the trait-mapping engine inside the scoring module 110. The database unit 120 supports version-controlled storage of all recommendation outputs generated by the artificial intelligence recommendation module 112, annotated with timestamps and revision history. The database unit 120 logs system feedback transactions handled by the feedback integration module 114 and documents remote clinical actions performed through the telemedicine integration module 116. The database unit 120 ensures data continuity and regulatory compliance, enabling future audits, population analytics, and research-based retrievals without compromising data privacy or user control.

The user device 122 functions as the primary personal interface for receiving user input and delivering system output and is operatively connected to the processing unit 106 through the communication network 104. The user device 122 includes hardware components such as touchscreens, biometric sensors, microphones, speakers, and cameras, enabling multimodal interaction with the health scoring and recommendation generation system. The user device 122 displays dashboard visualizations created by the dashboard module 118 and routes them to the user interface 124. The user device 122 receives questionnaire inputs, self-reported behaviors, and manual health log entries and transmits these to the data acquisition unit 102 for processing. The user device 122 receives live recommendations generated by the artificial intelligence recommendation module 112 and teleconsultation responses managed by the telemedicine integration module 116. The user device 122 also supports sensor-based data capture, such as passive monitoring of heart rate, movement, and temperature, and forwards those data streams to the feedback integration module 114. The user device 122 operates continuously while maintaining encrypted connection with the communication network 104. The user device 122 plays a critical role in enabling real-time updates, lifestyle monitoring, and remote medical interaction from any physical location.

The user interface 124 is disposed within the user device 122 and enables visual and interactive access to the system outputs, including health scores, recommendation pathways, profile data, and teleconsultation options. The user interface 124 supports structured visualization of graphs, scorecards, goal progress dashboards, and alert notifications generated by the dashboard module 118. The user interface 124 provides an interactive feedback panel allowing users to submit subjective data such as mood, stress levels, fatigue, or dietary preferences, which are transmitted to the data acquisition unit 102 for integration into future profiling and scoring operations. The user interface 124 presents recommendation content generated by the artificial intelligence recommendation module 112 in an organized and goal-specific layout that includes exercise routines, meal guidelines, recovery tasks, and mental wellness prompts. The user interface 124 includes live chat, video window, and appointment scheduling components for direct access to services facilitated by the telemedicine integration module 116. The user interface 124 receives updated score trends, benchmarking comparisons, and educational insights and displays them with navigation controls and personalization options. The user interface 124 ensures that the user experience is seamless, informative, and responsive to ongoing health monitoring and personalized recommendation delivery.

In one embodiment, data acquisition unit 102 further comprises a microbiome data interface that is receiving gut microbiome sequencing outputs and taxonomic profiles from external laboratory services and is transmitting the microbial diversity metrics through communication network 104 to processing unit 106 where health profiling module 108 is combining the microbial indicators with genomic, lifestyle, and physiological inputs to enrich the user profile, and scoring module 110 is integrating the microbial diversity metrics into composite health-related scores that are driving tailored nutrition recommendations generated by artificial intelligence recommendation module 112.

In one embodiment, data acquisition unit 102 further comprises a demographic and clinical history interface that is importing electronic medical record data including past diagnoses, medication regimens, and familial disease history and is forwarding the clinical history inputs through communication network 104 to processing unit 106 where health profiling module 108 is annotating the user profile with medical risk factors, and scoring module 110 is weighting the annotated clinical history data within polygenic risk analysis workflows and overall health score calculations to support refined risk stratification in telemedicine integration module 116.

In one embodiment, the system further comprises a privacy and consent management module that is operating alongside communication network 104 to enforce user consent directives and regulatory compliance during data transmission between data acquisition unit 102, processing unit 106, and database unit 120, and is logging consent records within database unit 120 to provide an auditable trail for telemedicine integration module 116 interactions and for maintaining secure handling of sensitive genomic and health information.

In one embodiment, processing unit 106 further comprises an environmental context recognition module that is receiving ambient condition data from sensors integrated in user device 122 and is relaying the environmental parameters through communication network 104 to health profiling module 108 and scoring module 110 where environmental modifiers are adjusting the weight of physiological and lifestyle inputs, and artificial intelligence recommendation module 112 is generating context-aware guidance that dynamically adapts exercise intensity, hydration recommendations, and recovery protocols based on real-time environmental factors.

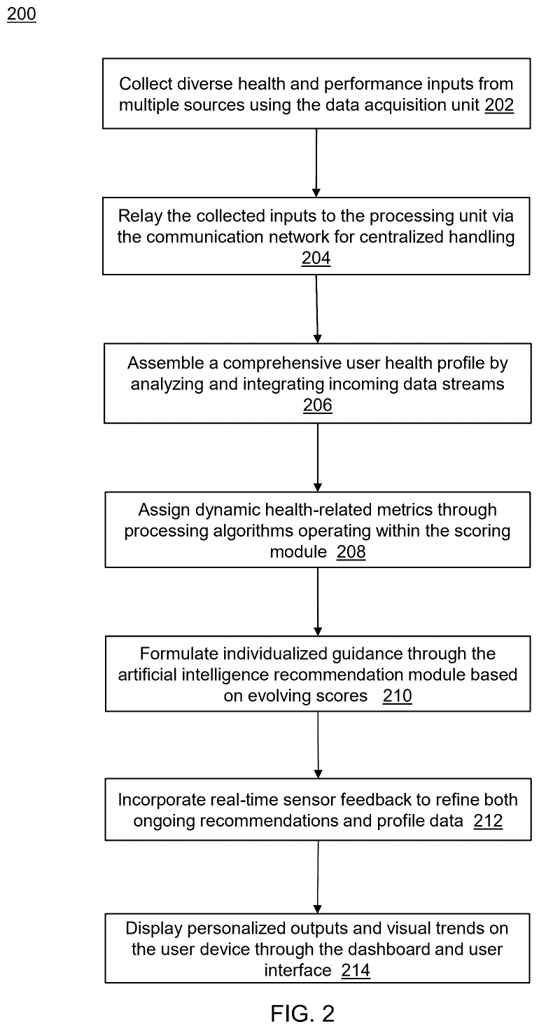

FIG. 2 illustrates a flowchart of a multi-modal health scoring and recommendation generation system, in accordance with an embodiment of the present invention.

At 202, collect diverse health and performance inputs from multiple sources using the data acquisition unit.

At 204, relay the collected inputs to the processing unit via the communication network for centralized handling.

At 206, assemble a comprehensive user health profile by analyzing and integrating incoming data streams.

At 208, assign dynamic health-related metrics through processing algorithms operating within the scoring module.

At 210, formulate individualized guidance through the artificial intelligence recommendation module based on evolving scores.

At 212, incorporate real-time sensor feedback to refine both ongoing recommendations and profile data.

At 214, display personalized outputs and visual trends on the user device through the dashboard and user interface.

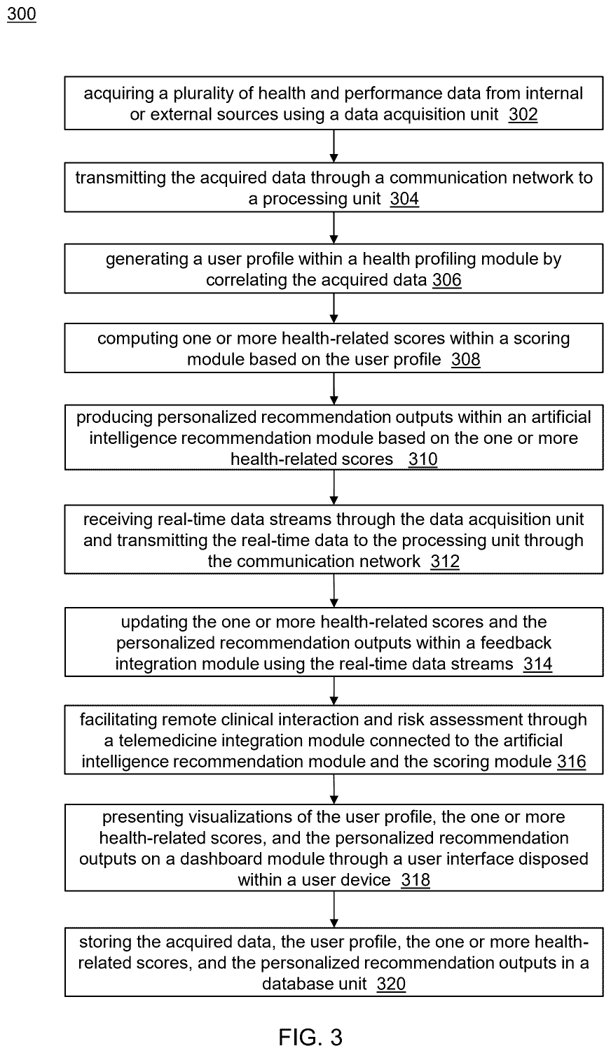

FIG. 3 illustrates a flowchart of a multi-modal health scoring and recommendation generation method, in accordance with an embodiment of the present invention.

At 302, acquiring a plurality of health and performance data from internal or external sources using a data acquisition unit.

At 304, transmitting the acquired data through a communication network to a processing unit.

At 306, generating a user profile within a health profiling module by correlating the acquired data.

At 308, computing one or more health-related scores within a scoring module based on the user profile.

At 310, producing personalized recommendation outputs within an artificial intelligence recommendation module based on the one or more health-related scores.

At 312, receiving real-time data streams through the data acquisition unit and transmitting the real-time data to the processing unit through the communication network.

At 314, updating the one or more health-related scores and the personalized recommendation outputs within a feedback integration module using the real-time data streams.

At 316, facilitating remote clinical interaction and risk assessment through a telemedicine integration module connected to the artificial intelligence recommendation module and the scoring module.

At 318, presenting visualizations of the user profile, the one or more health-related scores, and the personalized recommendation outputs on a dashboard module through a user interface disposed within a user device.

At 320, storing the acquired data, the user profile, the one or more health-related scores, and the personalized recommendation outputs in a database unit.

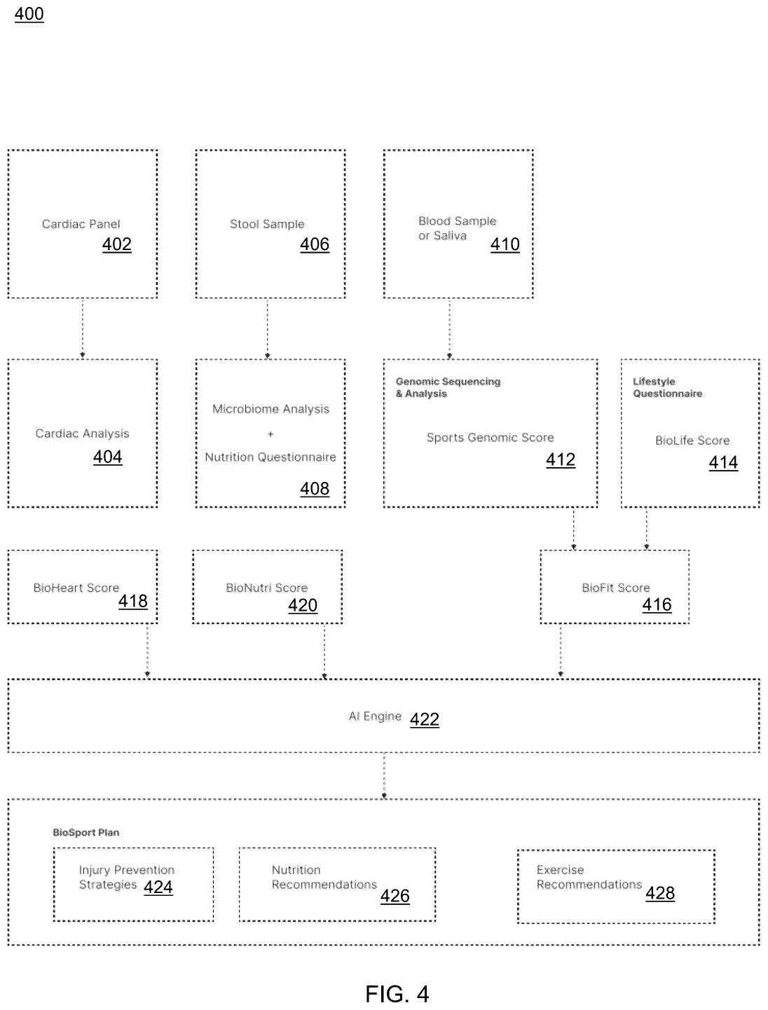

FIG. 4 illustrates a recommendation workflow of the health scoring and recommendation generation system, in accordance with an embodiment of the present invention.

The cardiac panel 402 collects genotype information linked to inherited cardiovascular conditions by using gene markers associated with cardiomyopathy, arrhythmias, and related syndromes. The cardiac panel 402 is used to extract raw data necessary for cardiac analysis 404, where the presence of these risk alleles is interpreted to estimate cardiac health risks. The cardiac panel 402 directly contributes to BioHeart score 418 through precise genomic profiling.

The cardiac analysis 404 interprets genetic variants received from the cardiac panel 402 using curated gene panels related to cardiomyopathy, rhythm disorders, and vascular abnormalities. The cardiac analysis 404 converts genetic input into quantifiable risk metrics used in BioHeart score 418. The cardiac analysis 404 transmits results through the artificial intelligence engine 422, allowing remote interpretation, scoring, and diagnostic input for sport-specific cardiac clearance planning.

The stool sample 406 is collected to extract gut microbiome profiles including bacterial composition, diversity indices, and presence of beneficial or harmful microbial species. The stool sample 406 is processed in microbiome analysis+nutrition questionnaire 408 to build dietary correlation models. The stool sample 406 provides raw ecological data to derive BioNutri score 420 and serves as one foundation for nutrition recommendations 426 in the final BioSport plan.

The microbiome analysis+nutrition questionnaire 408 performs microbial diversity interpretation and combines it with structured nutrition questionnaire responses to create a composite profile of the user's gut and dietary state. The microbiome analysis+nutrition questionnaire 408 calculates contribution weights for microbial resilience and nutrient assimilation, which feed into BioNutri score 420. This combination supports tailored outputs by artificial intelligence engine 422.

The blood sample or saliva 410 provides genomic material for whole genome sequencing and genotyping procedures, which are used by genomic sequencing and analysis systems to determine polygenic risk scores. The blood sample or saliva 410 is a key source for deriving sports genomic score 412. This biological material also supports calculations required for BioFit score 416 and the elite comparison benchmarking layer.

The sports genomic score 412 is calculated using genotypic data from blood sample or saliva 410, with a focus on performance traits such as endurance, muscle composition, VO2 max potential, and injury recovery capacity. The sports genomic score 412 is utilized in constructing BioFit score 416 and adjusting recommendation plans from artificial intelligence engine 422 including exercise recommendations 428.

The BioLife score 414 is generated from structured inputs received through a lifestyle questionnaire that collects information on physical activity, nutrition patterns, sleep behavior, substance use, stress levels, and other daily life variables. The BioLife score 414 directly contributes to the overall BioFit score 416, which is sent to the artificial intelligence engine 422 to contextualize non-genetic factors in the personalized outputs.

The BioFit score 416 serves as a central health and fitness index created by integrating sports genomic score 412, BioLife score 414, and additional inputs including age, sex, and lifestyle data. The BioFit score 416 is forwarded to the artificial intelligence engine 422, which uses the composite index to generate a custom BioSport plan containing injury prevention strategies 424, nutrition recommendations 426, and exercise recommendations 428.

The BioHeart score 418 is calculated using outputs from cardiac analysis 404. The BioHeart score 418 reflects the user's genetic risk for cardiovascular diseases and readiness for intense physical activity. The BioHeart score 418 is incorporated into the artificial intelligence engine 422 to generate cardiac-safe training and screening guidance used in the injury prevention strategies 424 component of the BioSport plan.

The BioNutri score 420 is derived from the integration of gut microbiome findings from stool sample 406 and lifestyle-based nutrition questionnaire data. The BioNutri score 420 is processed by the artificial intelligence engine 422 to determine suitable dietary adaptations. The BioNutri score 420 directly influences the recommendations delivered under nutrition recommendations 426 to ensure gut-aligned performance fueling.

The artificial intelligence engine 422 processes BioFit score 416, BioHeart score 418, and BioNutri score 420 using clustering logic, learned models, and phenotypic-genotypic comparison to elite athlete profiles. The artificial intelligence engine 422 generates contextual recommendations for athletic performance, nutrition, and health optimization. The artificial intelligence engine 422 delivers outputs to injury prevention strategies 424, nutrition recommendations 426, and exercise recommendations 428.

The injury prevention strategies 424 section of the BioSport plan includes genetically-informed and phenotype-adjusted recommendations that are processed by artificial intelligence engine 422 using risk profiles derived from BioHeart score 418 and musculoskeletal trait data embedded within sports genomic score 412. The injury prevention strategies 424 help minimize the user's susceptibility to stress fractures, joint overload, and muscle injuries.

The nutrition recommendations 426 are based on inputs processed through artificial intelligence engine 422 using BioNutri score 420 and dietary patterns derived from the microbiome analysis+nutrition questionnaire 408. The nutrition recommendations 426 include suggestions on macronutrient balance, nutrient timing, and food sensitivity management to support personalized athletic and metabolic goals.

The exercise recommendations 428 are generated by the artificial intelligence engine 422 using data from BioFit score 416 and sports genomic score 412. The exercise recommendations 428 define intensity, duration, and structure of physical activity tailored to the user's endurance profile, muscular response type, and recovery index. The exercise recommendations 428 align with elite benchmarking and cardiac safety requirements.

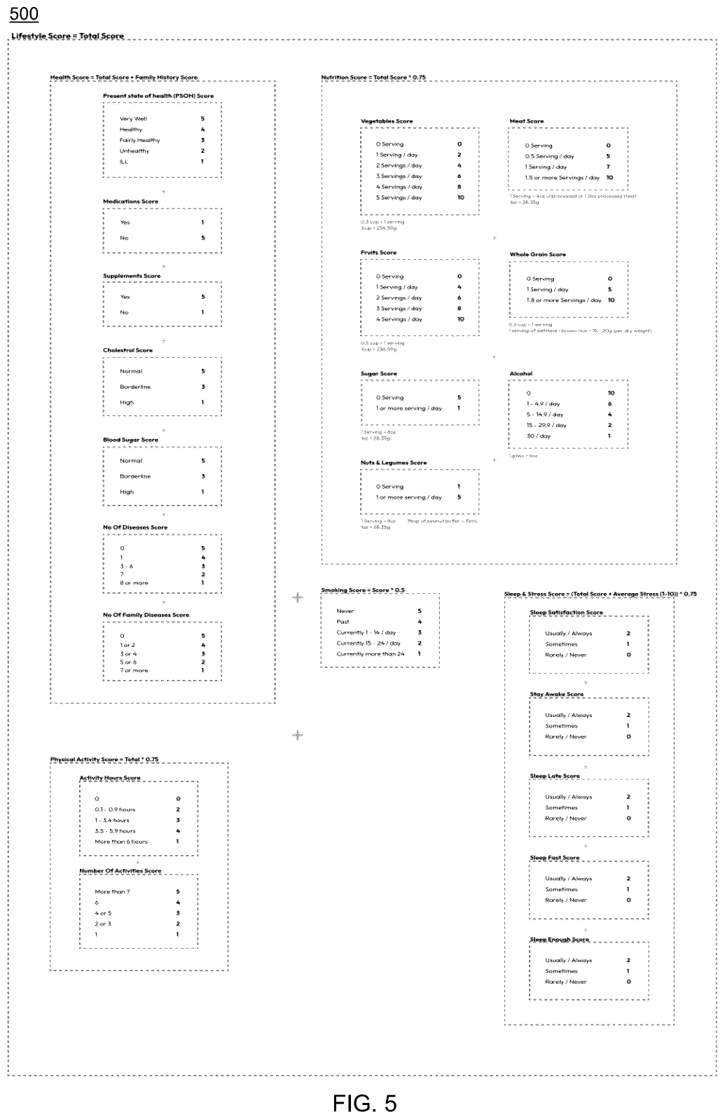

FIG. 5 illustrates a screenshot of the lifestyle score algorithm of a health scoring and recommendation generation system, in accordance with an embodiment of the present invention.

The lifestyle score algorithm integrates multiple sub-scores derived from health, nutrition, physical activity, smoking behavior, and sleep-stress domains. Each sub-score is calculated using structured inputs such as questionnaire responses and quantified behavior patterns, which are normalized to contribute to the total lifestyle score used in personalized recommendations.

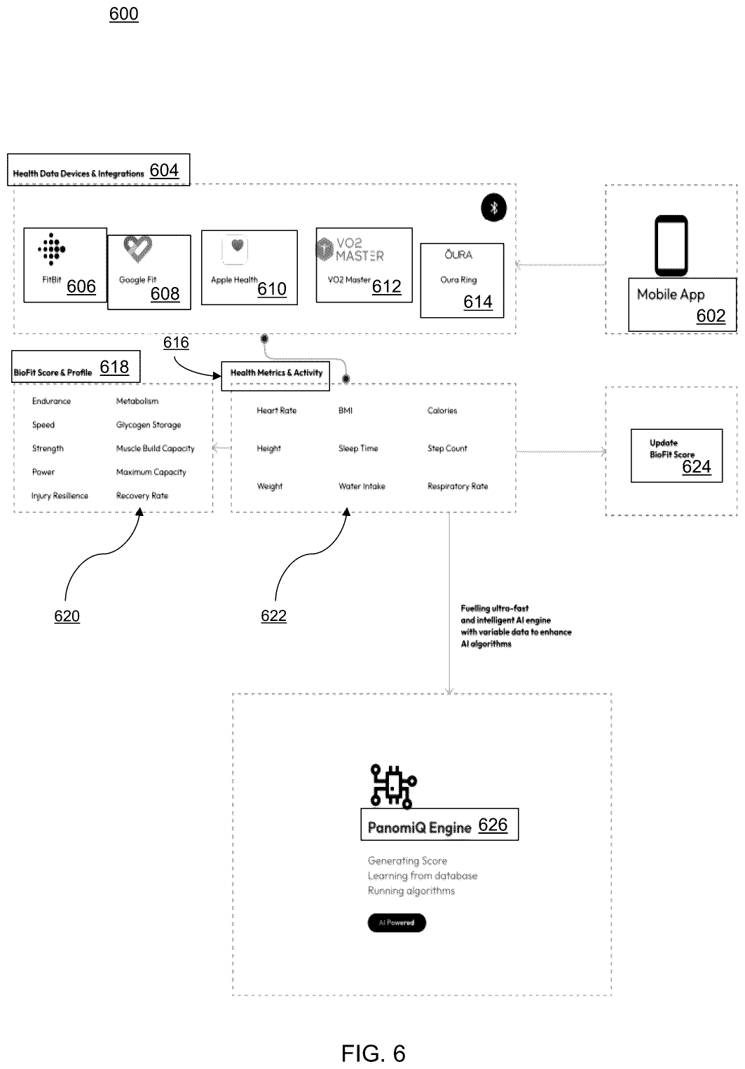

FIG. 6 illustrates a screenshot of the activity updating of a health scoring and recommendation generation system, in accordance with an embodiment of the present invention.

The mobile app 602 functions as the central interface for collecting, viewing, and transmitting user-specific health metrics and performance attributes to the health scoring and recommendation generation system. The mobile app 602 receives raw and processed data from wearable devices and displays interactive updates of BioFit score and recommendation changes in real time while maintaining connectivity with the update BioFit score 624 and PanomiQ engine 626.

The health data devices and integrations 604 serve as the aggregation layer for importing multi-dimensional physiological and activity metrics into the system. The health data devices and integrations 604 ensure real-time data flow between Fitbit 606, Google Fit 608, Apple Health 610, VO2 Master 612, and Oura Ring 614 and route the integrated signals to health metrics and activity 616. The health data devices and integrations 604 continuously sync and maintain bi-directional connectivity with the mobile app 602.

The Fitbit 606 device monitors movement, heart rate, and daily activity metrics and transmits the captured parameters into health metrics and activity 616. The Fitbit 606 directly influences water intake, sleep time, step count, and recovery-related inputs used to recalculate the BioFit score in update BioFit score 624 and PanomiQ engine 626.

The Google Fit 608 integration allows Android-based users to transmit physical activity patterns, step count, calorie burn, and biometric summaries into health metrics and activity 616. The Google Fit 608 system supports automated feedback to adjust scoring computations inside PanomiQ engine 626 and ensures consistent data streaming toward the mobile app 602 and update BioFit score 624.

The Apple Health 610 platform integrates with iOS-based user devices and gathers structured records on heart rate variability, respiratory rate, and calorie utilization. The Apple Health 610 synchronization feeds data directly into health metrics and activity 616 and dynamically updates endurance and metabolic scores within BioFit score and profile 618 via PanomiQ engine 626.

The VO2 Master 612 device captures oxygen consumption metrics and cardiorespiratory performance data and streams them to health metrics and activity 616. The VO2 Master 612 provides maximum capacity data that recalibrates endurance, strength, and cardiovascular thresholds within BioFit score and profile 618 using logic inside PanomiQ engine 626.

The Oura Ring 614 tracks sleep phases, body temperature, and readiness status and communicates the health status to health metrics and activity 616. The Oura Ring 614 enhances sleep time accuracy, influences water intake tracking, and aligns with recovery rate assessments inside BioFit score and profile 618, impacting recommendation pathways managed by PanomiQ engine 626.

The health metrics and activity 616 receives a continuous feed of physiological and behavioral data from all integrated health devices. The health metrics and activity 616 calculates attributes such as heart rate, body mass index, calories, height, sleep time, step count, weight, water intake, and respiratory rate and passes these into update BioFit score 624 and PanomiQ engine 626.

The BioFit score and profile 618 represent a composite metric of individual health and performance indicators based on device data, user inputs, and derived analytics. The BioFit score and profile 618 include endurance, speed, strength, power, injury resilience, metabolism, glycogen storage, muscle build capacity, maximum capacity, and recovery rate and form the core scoring basis updated via PanomiQ engine 626.

The performance traits 620 including endurance, speed, strength, power, injury resilience, metabolism, glycogen storage, muscle build capacity, maximum capacity, and recovery rate are dynamically updated from real-time metrics collected in health metrics and activity 616 and scored inside PanomiQ engine 626 to reflect physiological adaptation and health optimization levels.

The physiological measurements 622 including heart rate, body mass index, calories, height, sleep time, step count, weight, water intake, and respiratory rate are aggregated from Fitbit 606, Apple Health 610, Google Fit 608, VO2 Master 612, and Oura Ring 614 and influence updates to the BioFit score and profile 618 processed in PanomiQ engine 626.

The update BioFit score 624 continuously recalculates and refreshes the BioFit score based on incoming metrics from health metrics and activity 616. The update BioFit score 624 feeds recalculated values into PanomiQ engine 626 and pushes updated recommendations and insights to the mobile app 602 through the communication network and device integration layer.

The PanomiQ engine 626 acts as the centralized artificial intelligence infrastructure that generates BioFit scores, learns from continuously acquired data, and executes recommendation algorithms. The PanomiQ engine 626 processes variable inputs from BioFit score and profile 618 and health metrics and activity 616 and pushes precision recommendations to the mobile app 602 and update BioFit score 624.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A multi-modal health scoring and recommendation generation system comprising:
a data acquisition unit for acquiring a plurality of health and performance data from internal or external sources;
a communication network operatively connected to the data acquisition unit and configured for enabling data exchange between the data acquisition unit and other components of the system;
a processing unit operatively connected to the data acquisition unit through the communication network, the processing unit comprises:
a health profiling module configured for generating a user profile based on correlation of the acquired data;
a scoring module configured for generating one or more health-related scores based on the user profile;
an artificial intelligence recommendation module configured for producing personalized recommendation outputs;
a feedback integration module configured for updating the one or more health-related scores and the personalized recommendation outputs based on real-time data streams;
a telemedicine integration module configured for facilitating remote clinical interaction and risk assessment;
a dashboard module configured for generating interactive visualizations of the user profile, the one or more health-related scores, and the personalized recommendation outputs;
a database unit operatively connected to the processing unit and configured for securely storing the acquired data, the user profile, the one or more health-related scores, the personalized recommendation outputs, and related usage history;
a user device operatively connected to the processing unit through the communication network, the user device being configured for receiving user input and delivering output to the user;
a user interface disposed within the user device and configured for enabling access to visualizations, recommendations, and profile information.

2. The system of claim 1, wherein the data acquisition unit comprises a genomic data interface configured for receiving genomic data including whole genome sequencing or exome sequencing files from external laboratory systems.

3. The system of claim 1, wherein the data acquisition unit comprises a wearable device interface configured for receiving real-time activity and physiological data from one or more external wearable tracking devices.

4. The system of claim 1, wherein the processing unit further comprises a polygenic risk analysis module configured for generating a risk score for cardiac disease based on a plurality of genetic markers.

5. The system of claim 1, wherein the processing unit further comprises a benchmarking module configured for comparing the user profile with stored profiles of elite athletes.

6. The system of claim 1, wherein the health profiling module comprises a behavioral history analysis module configured for incorporating longitudinal lifestyle data into the user profile based on historical behavior patterns.

7. The system of claim 1, wherein the scoring module further comprises a trait-mapping engine for associating user data with performance, recovery, nutrition, and injury-resilience traits.

8. The system of claim 1, wherein the artificial intelligence recommendation module comprises a clustering logic engine for identifying user cohorts based on genomic and lifestyle similarity.

9. The system of claim 1, wherein the artificial intelligence recommendation module further comprises a rules-based inference engine trained on outcome data for generating risk mitigation suggestions.

10. The system of claim 1, wherein the feedback integration module further comprises a continuous data listener configured for detecting behavioural deviations from predicted activity patterns.

11. The system of claim 1, wherein the telemedicine integration module further comprises a triage prioritization engine for automatically classifying users based on risk levels for clinical review.

12. The system of claim 1, wherein the telemedicine integration module further comprises a secure interface for real-time voice, video, or text-based communication between the user and a remote healthcare provider.

13. The system of claim 1, wherein the dashboard module comprises a dynamic visualization builder configured for generating time-series trends for each score over a configurable time window.

14. The system of claim 1, wherein the user interface comprises an interactive feedback panel allowing users to submit responses or preferences for improving recommendation quality.

15. The system of claim 1, wherein the user device comprises a biometric sensor unit configured for locally capturing one or more physiological parameters including heart rate, skin temperature, or oxygen saturation.

16. The system of claim 1, wherein the database unit comprises a data encryption layer for secure long-term storage of genomic and health scoring information.

17. The system of claim 1, wherein the processing unit further comprises a learning module configured for adapting scoring and recommendation behaviour based on aggregated anonymous user data.

18. The system of claim 1, wherein the processing unit further comprises a context recognition module configured for adjusting recommendations based on environmental or temporal variables detected from the user device.

19. The multi-modal health scoring and recommendation generation method comprising:
acquiring a plurality of health and performance data from internal or external sources using a data acquisition unit;
transmitting the acquired data through a communication network to a processing unit;
generating a user profile within a health profiling module by correlating the acquired data;
computing one or more health-related scores within a scoring module based on the user profile;
producing personalized recommendation outputs within an artificial intelligence recommendation module based on the one or more health-related scores;
receiving real-time data streams through the data acquisition unit and transmitting the real-time data to the processing unit through the communication network;
updating the one or more health-related scores and the personalized recommendation outputs within a feedback integration module using the real-time data streams;
facilitating remote clinical interaction and risk assessment through a telemedicine integration module connected to the artificial intelligence recommendation module and the scoring module;
presenting visualizations of the user profile, the one or more health-related scores, and the personalized recommendation outputs on a dashboard module through a user interface disposed within a user device;
storing the acquired data, the user profile, the one or more health-related scores, and the personalized recommendation outputs in a database unit.

20. The method of claim 19, wherein the method further comprises the step of performing a benchmarking operation within the processing unit, wherein the benchmarking operation is comparing the user profile and the one or more health-related scores against stored profiles of elite performers to adjust the personalized recommendation outputs.
